(19) United States
(12) Patent Application Publication
(10) Pub. No.: US 2025/0176800 A1
(43) Pub. Date: Jun. 5, 2025

(54) ENDOSCOPE VACUUM VALVES WITH STRATEGIC RELATIVE PORT SIZING

(71) Applicant: Boston Scientific Scimed Inc., Maple Grove, MN (US)

(72) Inventors: Kurt Nicholas Robakiewicz, Upton, MA (US); Brian Luis, Boylston, MA (US)

(73) Assignee: Boston Scientific Scimed Inc., Maple Grove, MN (US)

(21) Appl. No.: 18/960,563

(22) Filed: Nov. 26, 2024

Related U.S. Application Data
(60) Provisional application No. 63/605,202, filed on Dec. 1, 2023.

(51) Int. Cl. A61B 1/00; A61B 1/015

(52) U.S. Cl. CPC A61B 1/00068; A61B 1/00105; A61B 1/015

(57) ABSTRACT

A suction valve for endoscopes (e.g., duodenoscopes) includes a valve housing with strategic relative port sizing to provide a vented configuration and a suction configuration. In the suction configuration, the endoscope conveys suction to a distal end of the endoscope's flexible tubular probe. In the vented configuration, the suction is mostly turned off at the distal end. The valve housing has a suction source opening leading to a vacuum pump, a vent opening that can be opened to atmosphere, and a probe opening leading to a suction tube in the flexible tubular probe. In some examples, the suction source opening is larger than the vent opening, which in turn is larger than the probe opening. With such port sizing, the suction valve can switch between the vented and suction configurations without relying on any moving parts. An operator can switch to the suction configuration by simply covering the vent opening.

### CROSS-REFERENCE TO RELATED APPLICATIONS

This application claims the benefit of U.S. Provisional Patent Application Ser. No. 63/605,202 filed on Dec. 1, 2023, the disclosures of which are incorporated herein by reference.

### TECHNICAL FIELD

Various aspects of this disclosure relate generally to endoscopes (e.g., duodenoscopes, colonoscopes, bronchoscopes, etc.) and more specifically to suction valves for endoscopes.

### BACKGROUND

Endoscopes enable medical practitioners to directly visualize internal cavities of patients without the need for invasive surgeries. Among the various types of endoscopes, duodenoscopes hold a prominent place due to their capability to explore the upper gastrointestinal tract, particularly the duodenum, pancreas, and bile ducts. Duodenoscopes facilitate not only visual examinations but also a range of therapeutic procedures, making them indispensable tools in modern medicine.

Duodenoscopes typically comprise a flexible tubular probe extending from a handle body. The probe is inserted into the patient, while the operator holds the handle body. A light source at a distal end of the probe provides illumination for viewing. Often a high-resolution camera is adjacent to the light source for capturing real-time images or videos of the internal cavities.

A duodenoscope's probe usually includes an internal working channel, allowing for the insertion of various instruments for procedures like biopsies, tissue removal, or stent placement. To provide maneuverability and access to intricate anatomical structures, many duodenoscope probes have internal wires. The tension in the wires can be adjusted in opposing sets of two by manipulating knobs on a handle body of the duodenoscope. Adjusting the wire tension enables bending and steering of the probe.

Handle bodies typically include a bifluidic valve (air/water valve) and a suction valve, which the operator controls manually. Bifluidic valves can be used for controlling fluid flow, such as air for insufflation and water for irrigation. Insufflation is a technique involving the introduction of air or carbon dioxide into the cavity being examined, which helps to expand the space, allowing for better visibility of the targeted area.

Irrigation typically involves the introduction of liquids, such as sterile water or saline. Irrigation can help clear blood, debris, or mucus from the visual field, ensuring clear visibility. Irrigation can also aid in therapeutic interventions by flushing out areas of interest, allowing for better access and manipulation of tissues.

Suction valves on the handle body are used by the operator to apply or terminate suction at the distal end of the probe. Suction valves often include a valve spool that can be moved manually between open and closed positions. When open, the applied suction can clear the probe's field of view by drawing fluid, tissue and other matter back through a suction tube within the probe. Moving the valve spool to the closed position terminates the suction. Suction valves typically include a spring for urging the valve spool to its closed position.

### SUMMARY

The present disclosure generally pertains to suction valves for controlling a fluid flowing through an endoscope that includes a flexible tubular probe extending from a handle body. In some examples, the endoscope is connectable to a suction source. In some examples, the suction valve is configurable selectively to a suction configuration and a vented configuration. Some examples of the suction valve include a valve housing supported by the handle body. Some examples of the suction valve include a probe opening with a first flow coefficient. In some examples the probe opening is connected in fluid communication with the flexible tubular probe. In some examples, the probe opening is at least partially defined by the valve housing. Some examples of the suction valve include a vent opening with a second flow coefficient. In some examples, the vent opening is in fluid communication with a surrounding atmosphere when the suction valve is in the vented configuration. In some examples, the vent opening is at least partially defined by the valve housing. In some examples, the vent opening is selectively open and closed. In some examples, the suction valve is in the vented configuration when the vent opening is open. In some examples, the suction valve is in the suction configuration when the vent opening is closed. Some examples of the suction valve further include a suction source opening with a third flow coefficient. In some examples, the suction source opening is connected in fluid communication with the suction source. In some examples, the suction source opening is defined by the valve housing. In some examples, the second flow coefficient is greater than the first flow coefficient and less than the third flow coefficient. In some examples, most of the fluid flowing through the suction valve flows from the vent opening to the suction source opening when the vent opening is open in the vented configuration. In some examples, most of the fluid flowing through the suction valve flows from the probe opening to the suction source opening when the vent opening is closed in the suction configuration.

In some examples, a suction valve includes a valve housing supported by the handle body. Some examples of the suction valve include a probe opening connected in fluid communication with the flexible tubular probe. In some examples, the probe opening is of a first size and at least partially defined by the valve housing. Some examples of the suction valve further includes a vent opening in fluid communication with surrounding atmosphere when the suction valve is in the vented configuration. In some examples, the vent opening is of a second size and at least partially defined by the valve housing. In some examples, the vent opening is selectively open and closed. In some examples, the suction valve is in the vented configuration when the vent opening is open. In some examples, the suction valve is in the suction configuration when the vent opening is closed. Some examples of the suction valve also include a suction source opening connected in fluid communication with the suction source. In some examples, the suction source opening is of a third size and at least partially defined by the valve housing. In some examples, the second size is larger than the first size and smaller than the third size. In some examples, most of the fluid flowing through the suction valve flows from the vent opening to the suction source opening when the vent opening is open in the vented configuration. In some examples, most of the fluid flowing through the suction valve flows from the probe opening to the suction source opening when the vent opening is closed in the suction configuration.

In some examples, a suction valve system includes a handle body of an endoscope. Some examples of the suction valve system include a valve housing supported by the handle body. In some examples, the valve housing at least partially defines a probe opening, a vent opening, and a suction source opening. In some examples, the probe opening is in fluid communication with the suction source opening and the vent opening in both the suction configuration and the vented configuration. In some examples, the vent opening is selectively open and closed. In some examples, the suction valve system is in the suction configuration when the vent opening is closed. In some examples, the suction valve system is in the vented configuration with the vent opening being in fluid communication with surrounding atmosphere when the vent opening is open. In some examples, the probe opening has a first flow coefficient. In some examples, the vent opening has a second flow coefficient when the vent opening is open. In some examples, the suction source opening has a third flow coefficient. In some examples, the second flow coefficient is greater than the first flow coefficient and less than the third flow coefficient. In some examples, most of the fluid flowing through the suction valve system flows from the vent opening to the suction source opening when the vent opening is open in the vented configuration. In some examples, most of the fluid flowing through the suction valve flows from the probe opening to the suction source opening when the vent opening is closed in the suction configuration. Some examples of the suction valve system also include an umbilicus attached to the handle body to connect a suction source to the suction source opening of the valve housing. Some examples of the suction valve system further include a flexible tubular probe connected to the handle body. Some examples of the suction valve system include a suction tube attached to the valve housing at the probe opening and extending through the flexible tubular probe.

The preceding summary is provided to facilitate an understanding of some of the features of the present disclosure and is not intended to be a full description. A full appreciation of the disclosure can be gained by taking the entire specification, claims, drawings and abstract as a whole.

### BRIEF DESCRIPTION OF THE DRAWINGS

FIG. 1 is a perspective view of an example endoscope with a suction valve incorporating various aspects of the present disclosure.

FIG. 2 is a cross-sectional view taken along line 2-2 of FIG. 1, wherein the suction valve 4 is in a vented configuration.

FIG. 3 is a cross-sectional view similar to FIG. 2 but showing the suction valve in a suction configuration.

FIG. 4 is a cross-sectional view similar to FIG. 2 but showing another example of the suction valve, wherein the suction valve is in a vented configuration.

FIG. 5 is a cross-sectional view similar to FIG. 4 but showing the suction valve in a suction configuration.

FIG. 6 is a cross-sectional view similar to FIG. 2 but showing another example of the suction valve, wherein the suction valve is in a vented configuration.

FIG. 7 is a cross-sectional view similar to FIG. 6 but showing the suction valve in a suction configuration.

FIG. 8 is a cross-sectional view similar to FIG. 2 but showing another example of the suction valve, wherein the suction valve is in a vented configuration.

FIG. 9 is a cross-sectional view similar to FIG. 8 but showing the suction valve in a suction configuration.

FIG. 10 is a cross-sectional view similar to FIG. 2 but showing another example of the suction valve, wherein the suction valve is in a vented configuration.

FIG. 11 is a cross-sectional view similar to FIG. 10 but showing the suction valve in a suction configuration.

FIG. 12 is an end view of an example suction valve with an example closure on an example valve housing, wherein the closure is fully open.

FIG. 13 is a cross-sectional view taken along line 13-13 of FIG. 12.

FIG. 14 is an end view similar to FIG. 12 but showing the closure partway open.

FIG. 15 is a cross-sectional view taken along line 15-15 of FIG. 14.

FIG. 16 is an end view similar to FIGS. 12 and 14 but showing the closure fully closed.

FIG. 17 is a cross-sectional view taken along line 17-17 of FIG. 16.

FIG. 18 is an end view of an example suction valve with an example closure on an example valve housing, wherein the closure is in a more-open position.

FIG. 19 is a cross-sectional view taken along line 19-19 of FIG. 18.

FIG. 20 is an end view similar to FIG. 18 but showing the closure partway open.

FIG. 21 is a cross-sectional view taken along line 21-21 of FIG. 20.

FIG. 22 is an end view similar to FIGS. 18 and 20 but showing the closure fully closed.

FIG. 23 is a cross-sectional view taken along line 23-23 of FIG. 22.

FIG. 24 is an end view of an example suction valve with an example closure on an example valve housing, wherein the closure is fully open.

FIG. 25 is a cross-sectional view taken along line 25-25 of FIG. 24.

FIG. 26 is an end view similar to FIG. 24 but showing the closure partway open.

FIG. 27 is a cross-sectional view taken along line 27-27 of FIG. 26.

FIG. 28 is an end view similar to FIGS. 24 and 26 but showing the closure fully closed.

FIG. 29 is a cross-sectional view taken along line 29-29 of FIG. 28.

FIG. 30 is an end view of an example suction valve with an example closure on an example valve housing, wherein the closure is fully open.

FIG. 31 is a cross-sectional view taken along line 31-31 of FIG. 30.

FIG. 32 is an end view similar to FIG. 30 but showing the closure partway open.

FIG. 33 is a cross-sectional view taken along line 33-33 of FIG. 32.

FIG. 34 is an end view similar to FIGS. 30 and 32 but showing the closure fully closed.

FIG. 35 is a cross-sectional view taken along line 35-35 of FIG. 34.

FIG. 36 is an end view of an example suction valve with an example closure on an example valve housing.

FIG. 37 is a cross-sectional view taken along line 37-37 of FIG. 36.

FIG. 38 is an end view of an example suction valve with a screen-style closure on an example valve housing.

FIG. 39 is a cross-sectional view taken along line 39-39 of FIG. 38.

### DESCRIPTION

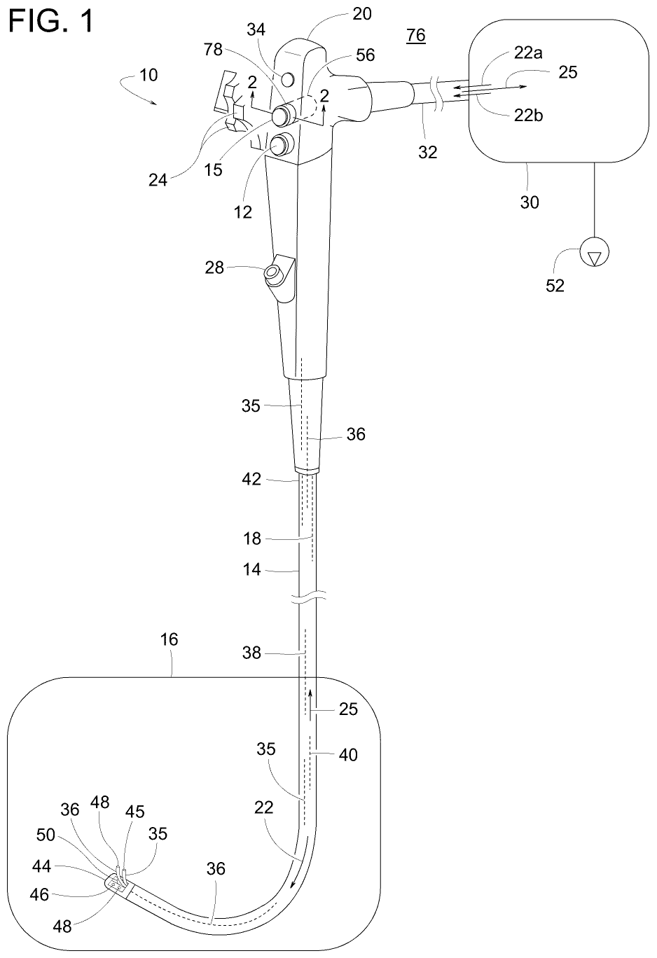

FIGS. 1-39 show various examples of a suction valve 15 (e.g., suction valves 15a-e) for an endoscope 10 and methods for using them. The term, “endoscope” represents any medical apparatus with a flexible tubular probe 14 for inserting into a patient 16 to visually explore the patient's internal tissues and cavities and to introduce or withdraw water, air, or other fluids when desired. Some example endoscopes 10 have internal wires 18 with adjustable tension for bending and steering the flexible tubular probe 14. Some examples of the endoscope 10, shown in FIG. 1, include duodenoscopes, colonoscopes, ureteroscopes, bronchoscopes, laparoscopes, sheaths, and catheters.

The endoscope 10 is illustrated as an example, so many of the following listed components are optional. Some examples of the endoscope 10 include components such as a handle body 20, the flexible tubular probe 14 extending from the handle body 20, the suction valve 15 for controlling the suction of a fluid 25 (e.g., bodily fluids) drawn back through the flexible tubular probe 14, a bifluidic valve 12 (air/water valve) for controlling the flow of a fluid 22 (e.g., a liquid 22a and a gas 22b), steering knobs 24 to adjust the tension in the internal wires 18, a biopsy port 28 for sampling withdrawn tissue or fluid, a control unit 30, an umbilicus 32 connecting the control unit 30 to the handle body 20, and an image capture button 34.

The control unit 30 and/or its associated external components provide various functions, such as supplying liquid 22a (e.g., water, saline, etc.), supplying gas 22b (e.g., air, carbon dioxide, etc.), providing a source of vacuum 52 (e.g., a vacuum pump, venturi, etc.), sending and receiving electrical signals, processing electrical signals, etc. Some of these functions are optional. The umbilicus 32 connects the control unit 30 in signal communication or fluid communication with the bifluidic valve 12, the suction valve 15, the flexible tubular probe 14, or other endoscope-related components. The term, “vacuum” means anything less than atmospheric pressure (i.e., less than 14.7 psia).

In some examples, the flexible tubular probe 14 contains various components such as the internal wires 18 for steering, tubing 36 (one or more tubes) for conveying fluids 22, a suction tube 35 for drawing the fluid 25 from the patient 16, a fiber optic cable 38 for conveying images or light, and electrical wires 40 for conveying electrical power or signals. Some of these probe components are optional.

The flexible tubular probe 14 has a proximal end 42 and a distal end 44. The proximal end 42 connects to the handle body 20, and the distal end 44 extends away from the handle body 20. At the distal end 44, some examples of the flexible tubular probe 14 include a light 46 (or fiber optic cable leading thereto) for illuminating a patient's internal cavities, a camera 47 (or fiber optic cable leading thereto), a tip 45 of the suction tube 35, a tip 48 of the tubing 36, and an elevator 50 for tilting tips 45 and/or 48. The elevator 50 is also known as a swing stand, a pivot stand, and a raising bed.

The tip 48 of the tubing 36 is open to deliver fluid 22 into the patient 16 for insufflating or irrigating, while the tip 45 of the suction tube 35 is open to draw fluids 25 from within the patient 16. In some examples, the source of vacuum 52 draws the fluid 25 in series from the patient 16, through the open tip 45, through the suction tube 35, through the suction valve 15, through the umbilicus 32, and out through the control unit 30.

An operator 62 pressing or, in some examples, just placing their finger on the top end of the suction valve 15 places the suction valve 15 in a suction configuration to apply appreciable suction to the suction tube 35. The operator 62 removing their finger returns the suction valve 15 to a vented configuration, alleviating the vacuum in the suction tube 35. The suction valve 15 thus provides a simple way of turning the suction on and off at the tip 45 of the suction tube 35.

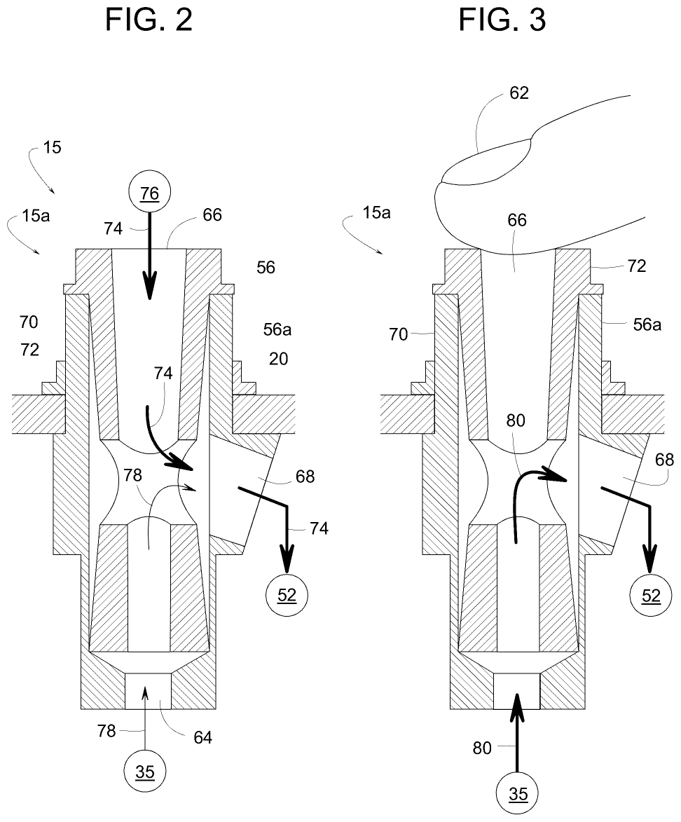

FIGS. 2 and 3 show the suction valve 15a in a vented configuration (FIG. 2) and a suction configuration (FIG. 3). In this example, suction valve 15a comprises a valve housing 56 (e.g., valve housings 56a-e) attached to the handle body 20. In some examples, the suction valve 15a at least partially defines a probe opening 64, a vent opening 66, and a suction source opening 68. The probe opening 64 connects to the suction tube 35 in the flexible tubular probe 14. The suction source opening 68 connects to the suction source 52 via the umbilicus 32. The vent opening 66 is normally open and in fluid communication with the surrounding atmosphere 76, as shown in FIG. 2. The operator 62, however, can close the vent opening 66 with their finger, as shown in FIG. 3. The terms, “surrounding atmosphere” and “local atmosphere” refer to the room air to which the exterior of the handle body 20 is exposed.

To achieve desired flow patterns through the suction valve 15a, the relative sizes and/or shapes of the probe opening 64, the vent opening 66 and the suction source opening 68 are strategically designed. In some examples, the suction source opening 68 is larger than the vent opening 66, and the vent opening 66, in turn, is larger than the probe opening 64. The relative sizes, in some examples, provides the probe opening 64 with a first flow coefficient, the vent opening 66 with a second flow coefficient, and the suction source opening 68 with a third flow coefficient, wherein the second flow coefficient is greater than the first flow coefficient and less than the third flow coefficient.

The term “size,” as it pertains to the size of an opening, is the magnitude of the smallest cross-sectional area of the opening, wherein the cross-sectional area is perpendicular to the direction of fluid flowing through the opening. In examples where the opening is cylindrical, the size of such an opening equals the cylinder's diameter squared multiplied by pi divided by four. In examples where the opening converges from a larger diameter to a smaller diameter, the size of such an opening equals the smaller diameter squared multiplied by pi divided by four. In examples where an opening comprises a plurality of apertures, the size of such an opening is the cumulative cross-sectional area of the opening's plurality of apertures.

The term, “flow coefficient’ (abbreviated Cv) is a dimensionless value known as a relative measure of how well a valve, conduit, orifice or other fluid-conveying element can pass a fluid. Larger flow coefficients means more fluid can pass for a given pressure drop. In some examples, a flow coefficient of one means that a valve will pass one gallon per minute of water at sixty degrees Fahrenheit with a pressure drop of one psig.

Some examples of the valve housing 56 comprise an outer shell 70 and a valve cartridge 72. In some examples, the valve cartridge 72 is axisymmetrical about its longitudinal axis, so it can be installed in any rotational orientation. The valve cartridge 72, in some examples, connects the probe opening 64 in fluid communication with both the suction source opening 68 and the vent opening 66 regardless of whether the suction valve 15a is in its vented configuration or suction configuration.

With the second flow coefficient of the vent opening 66 being greater than the first flow coefficient of the probe opening 64 and less than the third flow coefficient of the suction source opening 68, the suction source opening 68 has a much greater tendency to draw air from the vent opening 66 than from the probe opening 64 when the vent opening 66 is uncovered in the vented configuration, as shown in FIG. 2. So when the vent opening 66 is uncovered, most of the air flowing through the suction valve 15a flows from the vent opening 66 to the suction source opening 68. This is depicted in FIG. 2 with the bold arrows 74 indicating greater airflow than the thin arrows 78. With most of the airflow going from the vent opening 66 to the suction source opening 68, only an inconsequential amount of suction occurs the tip 45 of the suction tube 35.

Closing the vent opening 66, however, places the suction valve 15a in the suction configuration, as shown in FIG. 3. In the suction configuration, essentially all of the flow through the suction valve 15a is from the probe opening 64 to the suction source opening 68, as indicated by the bold arrows 80. Thus, an appreciable amount of suction develops at the tip 45 of the suction tube 35 in the suction configuration.

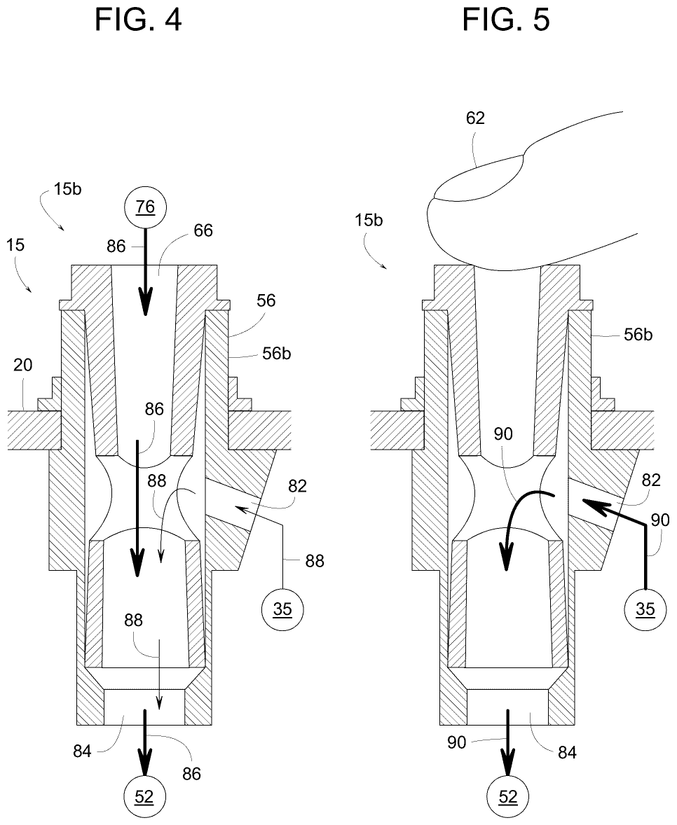

In some examples, the locations of the suction source opening 68 and the probe opening 64 are reversed. FIGS. 4 and 5, for example, show the suction valve 15b comprising the valve housing 56b with a probe opening 82 along the side and a suction source opening 84 at the bottom. The relative size and flow coefficients of the probe opening 82, the vent opening 66, and the suction source opening 84 are basically the same as the corresponding openings 64, 66 and 68 of the suction valve 15a.

FIG. 4 shows the suction valve 15b in the vented configuration, and FIG. 5 shows the suction configuration. In the vented configuration, most of the flow tends to go from the vent opening 66 to the suction source opening 84, as depicted by the bold arrows 86. Only a slight amount of flow goes from the probe opening 35 to the suction source opening 52, as indicated by the thin arrows 88. In the vented configuration, only an inconsequential amount of suction develops at the tip 45 of the suction tube 35.

Closing the vent opening 66, however, places the suction valve 15b in the suction configuration, as shown in FIG. 5. In the suction configuration, essentially all of the flow through the suction valve 15b is from the probe opening 82 to the suction source opening 84, as indicated by the bold arrows 90. Thus, an appreciable amount of suction develops at the tip 45 of the suction tube 35 in the suction configuration.

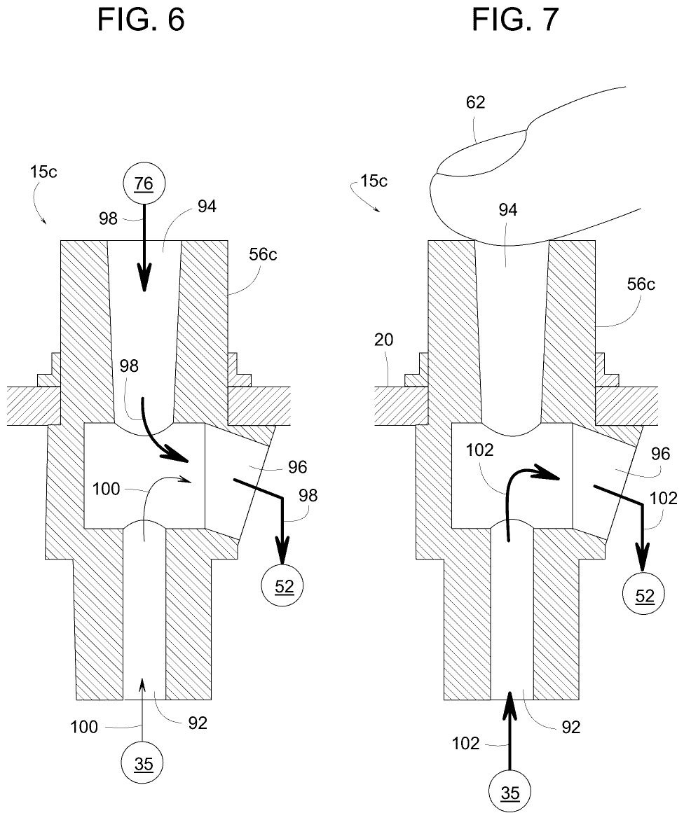

FIGS. 6 and 7 show the suction valve 15c, which is similar to suction valve 15a. The suction valve 15c, however, includes a single monolithic valve housing 56c (seamless unitary piece), instead of a valve housing comprised of two parts (e.g., the outer shell 70 and the valve cartridge 72 of the suction valve 15a).

The valve housing 56c at least partially defines a probe opening 92, a vent opening 94 and a suction source opening 96. The openings 92, 94, and 96 correspond in size and/or flow coefficients with the openings 64, 66, and 68 respectively.

FIG. 6 shows the suction valve 15c in the vented configuration, and FIG. 7 shows the suction configuration. In the vented configuration, most of the flow tends to go from the vent opening 94 to the suction source opening 96, as depicted by bold arrows 98. Only a slight amount of flow goes from the probe opening 92 to the suction source opening 96, as indicated by thin arrows 100. In the vented configuration, only an inconsequential amount of suction develops at the tip 45 of the suction tube 35.

Closing the vent opening 94, as shown in FIG. 7, places the suction valve 15b in the suction configuration. In the suction configuration, essentially all of the flow through the suction valve 15c is from the probe opening 92 to the suction source opening 96, as indicated by bold arrows 102. Thus, an appreciable amount of suction develops at the tip 45 of the suction tube 35 in the suction configuration.

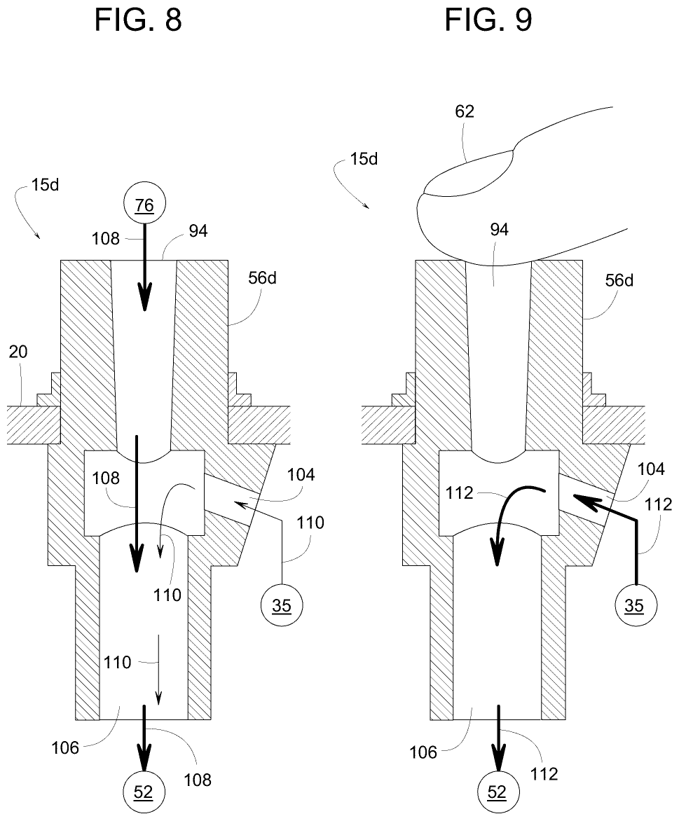

In some examples, the locations of the suction source opening 96 and the probe opening 92 are reversed. FIGS. 8 and 9, for example, show the suction valve 15d comprising a valve housing 56d with a probe opening 104 along the side and a suction source opening 106 at the bottom. The relative size and flow coefficients of the probe opening 104, the vent opening 94, and the suction source opening 106 are basically the same as the corresponding openings 92, 94 and 96 of the suction valve 15c.

FIG. 8 shows the suction valve 15d in the vented configuration, and FIG. 9 shows the suction configuration. In the vented configuration, most of the flow tends to go from the vent opening 94 to the suction source opening 106, as depicted by bold arrows 108. Only a slight amount of flow goes from the probe opening 104 to the suction source opening 106, as indicated by thin arrows 110. In the vented configuration, only an inconsequential amount of suction develops at the tip 45 of the suction tube 35.

Closing the vent opening 94, however, places the suction valve 15d in the suction configuration, as shown in FIG. 9. In the suction configuration, essentially all of the flow through the suction valve 15d is from the probe opening 104 to the suction source opening 106, as indicated by bold arrows 112. Thus, an appreciable amount of suction develops at the tip 45 of the suction tube 35 in the suction configuration.

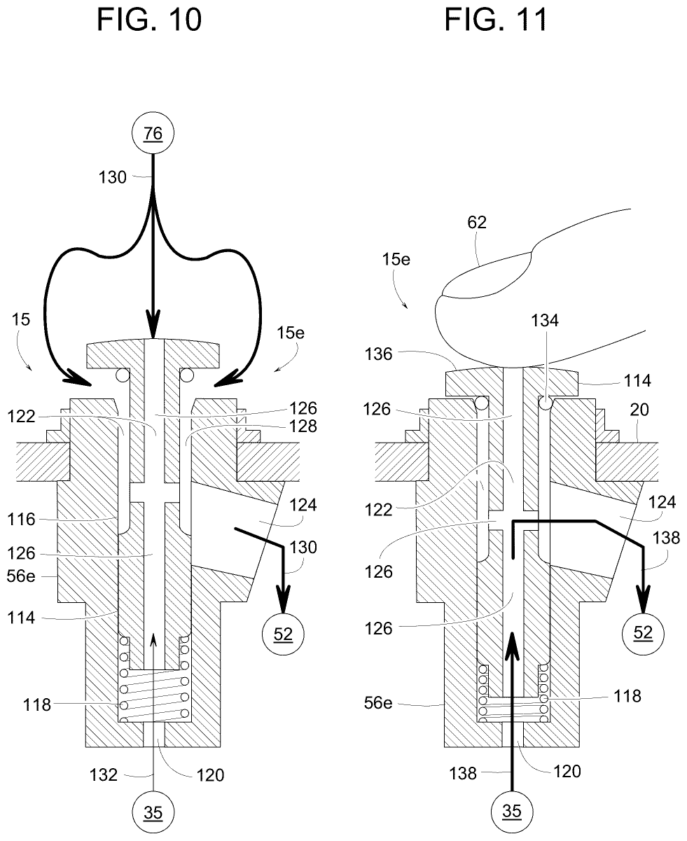

The suction valve 15e, shown in FIGS. 10 and 11, is similar to the suction valve 15c. A main difference, however, is that suction valve 15e includes a valve spool 114 installed within a central bore 116 of the valve housing 56e. The valve spool 114 is movable from a home position (FIG. 10) to a depressed position (FIG. 11). In some examples, a spring 118 urges the valve spool 114 to its home position. The valve spool's movement provides the operator 62 with familiar tactile feedback indicating when the suction valve 15e is in the suction configuration, as shown in FIG. 11. FIG. 10 shows the suction valve 15e in the vented configuration.

In some examples, the valve housing 56e at least partially defines a probe opening 120, a vent opening 122, and a suction source opening 124. The probe opening 120 connects to the suction tube 35 in the flexible tubular probe 14. The suction source opening 124 connects to the suction source 52 via the umbilicus 32. The vent opening 122 is normally open and in fluid communication with the surrounding atmosphere 76 when the suction valve 15e is in the vented configuration, as shown in FIG. 10.

In some examples, the valve spool 114 has a valve spool passageway 126 that connects the suction source opening 124 in fluid communication with the probe opening 120. In the vented configuration, the valve spool passageway 126 is also in fluid communication with the surrounding atmosphere 76.

To achieve desired flow patterns through the suction valve 15e, the relative sizes and/or shapes of the probe opening 120, the vent opening 122 and the suction source opening 124 are strategically designed. In some examples, the suction source opening 124 is larger than the vent opening 122, and the vent opening 122, in turn, is larger than the probe opening 120. The relative sizes, in some examples, provides the probe opening 120 with a first flow coefficient, the vent opening 122 with a second flow coefficient, and the suction source opening 124 with a third flow coefficient, wherein the second flow coefficient is greater than the first flow coefficient and less than the third flow coefficient.

In the illustrated example, the vent opening 122 is comprised of two paths. A first path is provided by the valve spool passageway 126 in the valve spool 114. A second path is provided an annular gap 128 between the valve spool 114 and the central bore 116 of the valve housing 56e. The vent opening's flow coefficient takes a cumulative account of both the valve spool passageway 126 and the annular gap 128.

With the second flow coefficient of the vent opening 122 being greater than the first flow coefficient of the probe opening 120 and less than the third flow coefficient of the suction source opening 124, the suction source opening 124 has a much greater tendency to draw air from the vent opening 122 than from the probe opening 120 when the valve spool 114 is in the home position with the vent opening 122 uncovered in the vented configuration, as shown in FIG. 10. In the vented configuration, most of the air flowing through the suction valve 15e flows from the vent opening 122 to the suction source opening 124. This is depicted by bold arrows 130 in FIG. 10. Due to the relatively small size of the probe opening 120, only a slight amount of flow goes from the probe opening 120 to the suction source opening 124, as indicated by thin arrow 132 in FIG. 10. With most of the airflow going from the vent opening 122 to the suction source opening 124 in the vented configuration, only an inconsequential amount of suction develops at the tip 45 of the suction tube 35.

Pressing the valve spool 114 from its home position (FIG. 10) to its depressed position (FIG. 11) closes the vent opening 122 to place the suction valve 15e in the suction configuration, as shown in FIG. 11. In some examples, a seal 134 (e.g., O-ring, gasket, etc.) ensures a positive seal between the valve housing 56e and a pushbutton head 136 of the valve spool 114.

In the suction configuration, essentially all of the flow through the suction valve 15e goes from the probe opening 120 to the suction source opening 124, as indicated by bold arrows 138 in FIG. 11. This causes an appreciable amount of suction to develop at the tip 45 of the suction tube 35 in the suction configuration.

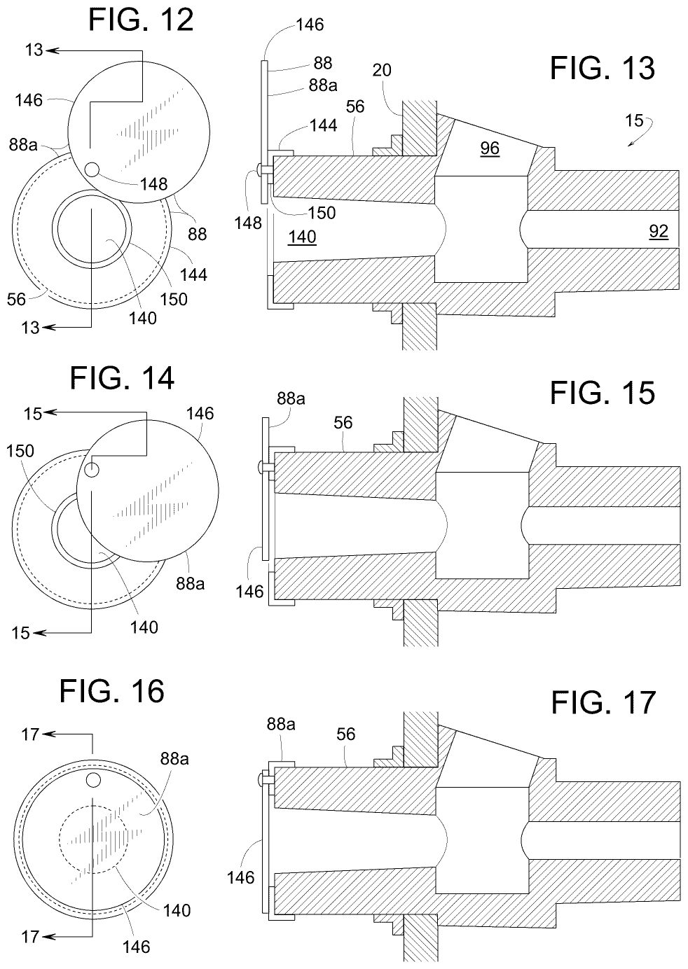

FIGS. 12-39 show various closures 88 (e.g., closures 88a-f) for fully blocking, partially obstructing, and/or adjusting a vent opening 140 of a valve housing 56. For sake of example, the valve housing 56 is similar to the valve housing 56c of FIGS. 6 and 7; however, the various closures 88a-f can be applied to any of the valve housings shown in FIGS. 1-37.

In the example shown in FIGS. 12-17, the closure 88a includes a base 144, a cover 146 and a pin 148. The base 144 has an aperture 150 and connects to the valve housing 56 at the vent opening 140. The pin 148 pivotally connects the cover 146 to the base 144. The pin 148 allows the cover 146 to be swung to a fully open or less-closed position (FIGS. 12 and 13), a more-closed position (FIGS. 16 and 17), and various partially open positions (e.g., FIGS. 14 and 15). The cover's range of positions provide means for varying the flow coefficient of the vent opening 140, thereby adjusting the amount of suction applied to the tip 45 of the suction tube 35.

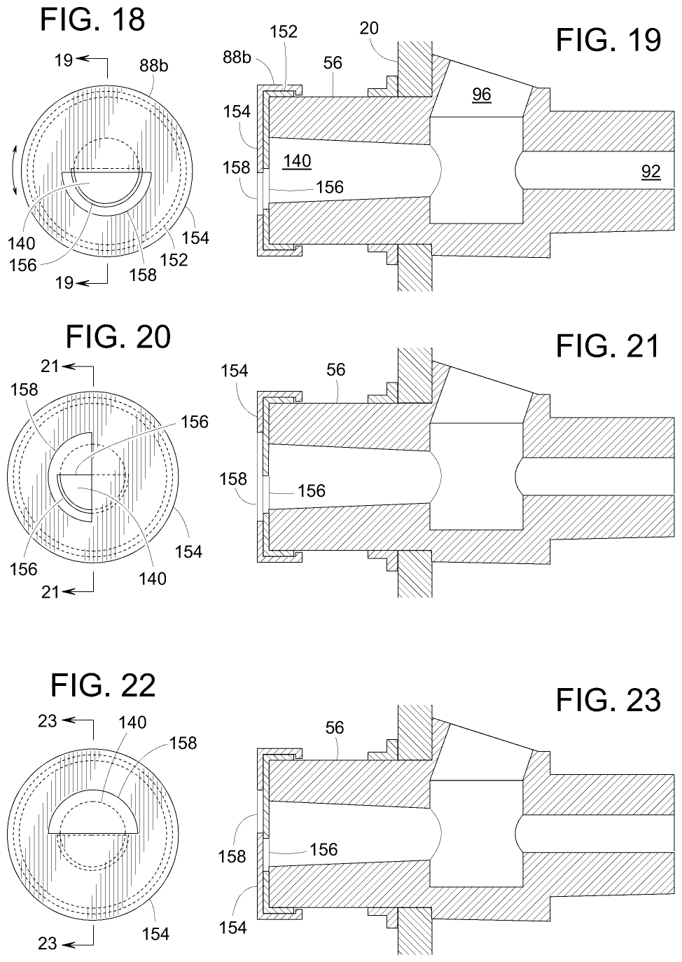

In the example shown in FIGS. 18-23, the closure 88b includes a base 152 and a cover 154. The base 152 has a base aperture 156 and connects to the valve housing 56 at the vent opening 140. The cover 154 has a cover aperture 158 and can rotate relative to the base 152 to adjust the registry of the two apertures 156 and 158. The cover 154 can be rotated to a more-open position (FIGS. 18 and 19), a fully closed or more-closed position (FIGS. 22 and 23), and various partially open positions (e.g., FIGS. 20 and 21). The cover's ability to rotate provides means for varying the flow coefficient of the vent opening 140, thereby adjusting the amount of suction applied to the tip 45 of the suction tube 35.

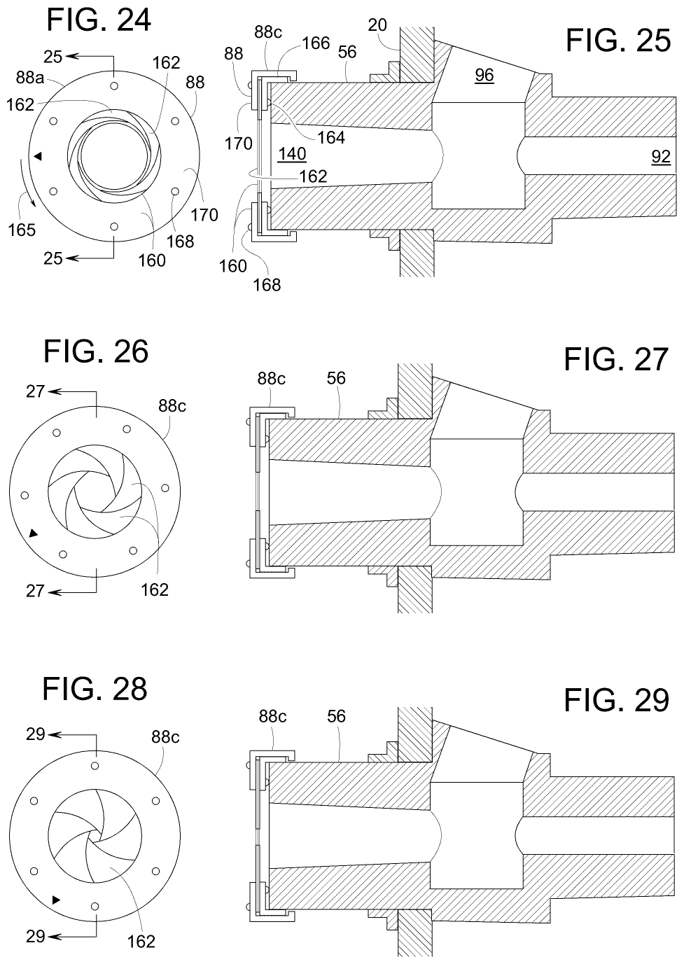

In the example shown in FIGS. 24-29, the closure 88c includes an iris valve 160 comprising a plurality of leaves 162. A lower set of pins 164 connect the leaves 162 to a lower ring 166, and an upper set of pins 168 connect the leaves 162 to an upper ring 170. The lower ring 166 is affixed to the valve housing 56. The upper ring 170 can rotate relative to the lower ring 166. Rotating 165 the upper ring 170 causes the leaves 162 to pivot about the upper and lower set of pins 164 and 168. As the leaves 162 pivot, they move radially inward or outward, depending on the upper ring's direction of rotation. The leaves 162 can move to a fully open or less-closed position (FIGS. 24 and 25), a fully or more-closed position (FIGS. 28 and 29), and various partially open positions (e.g., FIGS. 26 and 27). The range of leaf positions provide the iris valve 160 with means for varying the flow coefficient of the vent opening 140, thereby adjusting the amount of suction applied to the tip 45 of the suction tube 35.

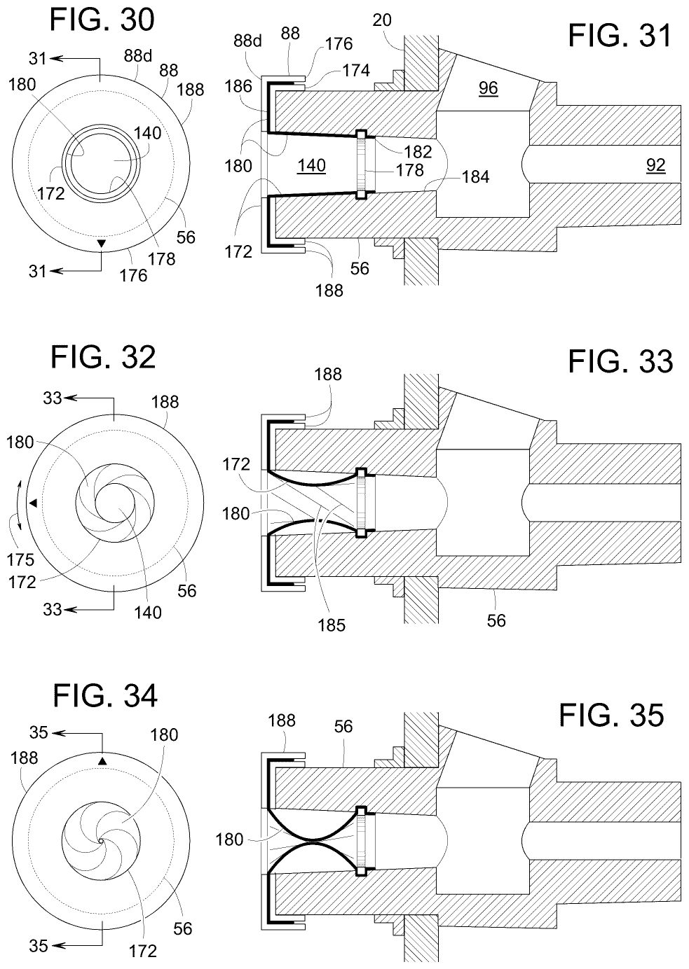

In the example shown in FIGS. 30-35, the closure 88d includes an iris valve 172 of another style. In this example, the closure 88d comprises a lower ring 174, an upper ring 174, a circular retainer 178, and a tubular diaphragm 180. In some examples, the diaphragm 180 is a thin, elastic monolithic sheet of material in a seamless tubular form. Example materials of the tubular diaphragm 180 include latex, elastane (e.g., Spandex), silicone, nitrile, neoprene, etc. The circular retainer 178 affixes a lower end 182 of the diaphragm 180 to an inner bore 184 of the valve housing 56. An upper end 186 of the diaphragm 180 is clamped between the upper and lower rings 174 and 176. The upper and lower rings 174 and 176 are stationary relative to each other but can rotate as a ring assembly 188 relative to the valve housing 56.

Rotating the ring assembly 188 twists the diaphragm 180 to varying degrees, depending on the degree of rotation. In some examples, the twisting action transforms the diaphragm 180 from a frustoconical shape (FIG. 31) into the shape of an approximate one-sheet hyperboloid, as shown in FIGS. 33 and 35. Crease lines 185 will naturally occur upon twisting the diaphragm 180. In some examples, the ring assembly 188 can be rotated over a range of zero to 180 degrees. Zero-degrees is shown in FIGS. 30 and 31, ninety-degrees is shown in FIGS. 32 and 33, and 180-degrees is shown in FIGS. 34 and 35.

As can be seen in FIGS. 30-35, the iris valve 172 can be adjusted to a fully open or less-closed position (FIGS. 30 and 31), a fully or more-closed position (FIGS. 34 and 35), and various partially open positions (e.g., FIGS. 32 and 33). Twisting the diaphragm 180 provides the iris valve 172 with means for varying the flow coefficient of the vent opening 140, thereby adjusting the amount of suction applied to the tip 45 of the suction tube 35.

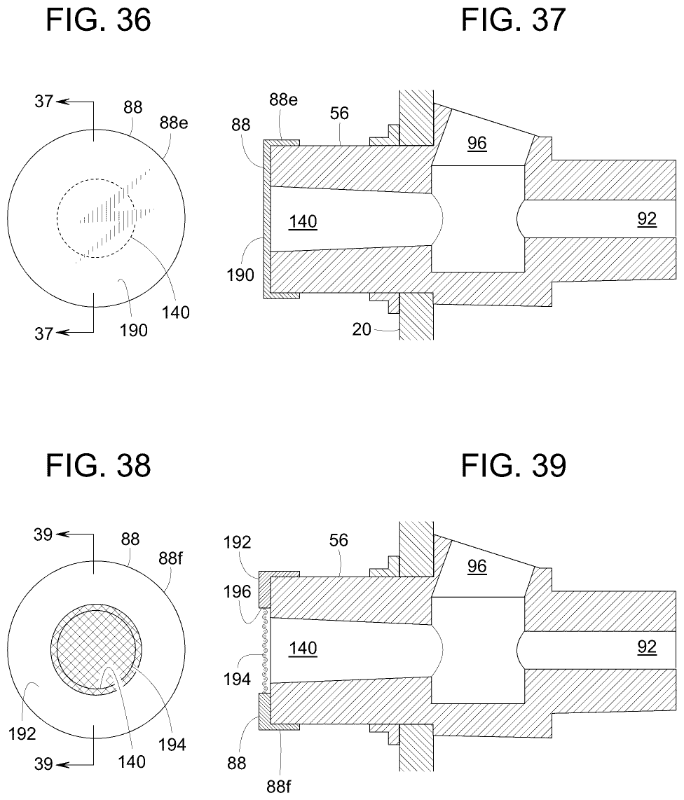

In the example shown in FIGS. 36 and 37, the closure 88e is a readily removable cover 190 attachable to the valve housing 56 to selectively cover or uncover the vent opening 140. Example means for holding the closure 88e in place include a snap connection, a press-fit, magnets, etc.

In the example shown in FIGS. 38 and 39, the closure 88f is a cover 192 with a screen 194 or filter extending across an aperture 196 in the cover 192. The screen 194 helps prevent debris or other contamination from entering the valve housing 56. Selectively installing or removing the closure 88f can modify the flow coefficient of the vent opening 140.

Some examples of the suction valve 15a-e (FIGS. 1-39) can be defined as described in the following examples 1-20.

Example-1 A suction valve for controlling a fluid flowing through an endoscope that includes a flexible tubular probe extending from a handle body, the endoscope being connectable to a suction source, the suction valve being configurable selectively to a suction configuration and a vented configuration, the suction valve comprising: a valve housing to be supported by the handle body; a probe opening with a first flow coefficient to be connected in fluid communication with the flexible tubular probe, the probe opening being at least partially defined by the valve housing; a vent opening with a second flow coefficient in fluid communication with a surrounding atmosphere when the suction valve is in the vented configuration, the vent opening being at least partially defined by the valve housing, the vent opening being selectively open and closed, the suction valve being in the vented configuration when the vent opening is open, the suction valve being in the suction configuration when the vent opening is closed, and a suction source opening with a third flow coefficient to be connected in fluid communication with the suction source, the suction source opening being defined by the valve housing, the second flow coefficient being greater than the first flow coefficient and less than the third flow coefficient, most of the fluid flowing through the suction valve flows from the vent opening to the suction source opening when the vent opening is open in the vented configuration, most of the fluid flowing through the suction valve flows from the probe opening to the suction source opening when the vent opening is closed in the suction configuration.

Example-2 The suction valve of Example-1, wherein the probe opening has a first minimum diameter, the vent opening has a second minimum diameter, the suction source opening has a third minimum diameter, and the second minimum diameter is larger than the first minimum diameter and smaller than the third minimum diameter.

Example-3 The suction valve of Example-1, wherein the second flow coefficient is closer in value to the third flow coefficient than to the first flow coefficient.

Example-4 The suction valve of Example-1, wherein the suction valve remains totally stationary relative to the handle body as the suction valve changes between the suction configuration and the vented configuration.

Example-5 The suction valve of Example-1, wherein the probe opening remains open in both the vented configuration and the suction configuration.

Example-6 The suction valve of Example-1, wherein the probe opening is connected in fluid communication with both the suction source opening and the vent opening in both the vented configuration and the suction configuration.

Example-7 The suction valve of Example-1, wherein the valve housing comprises an outer shell containing a valve cartridge, and the valve cartridge connects the probe opening in fluid communication with both the suction source opening and the vent opening in both the vented configuration and the suction configuration.

Example-8 The suction valve of Example-7, wherein the valve cartridge remains stationary relative to the outer shell as the suction valve changes between the suction configuration and the vented configuration.

Example-9 The suction valve of Example-7, wherein the valve cartridge is axisymmetrical.

Example-10 The suction valve of Example-1, further comprising a closure attached to the valve housing at the vent opening.

Example-11 The suction valve of Example-10, wherein the closure is movable between a more-closed position and an less-closed position, the closure providing a greater obstruction to the vent opening in the more-closed position than in the less-closed position.

Example-12 The suction valve of Example-11, wherein at least part of the closure is rotatable between the more-closed position and the less-closed position.

Example-13 The suction valve of Example-12, wherein the closure is an iris valve.

Example-14 The suction valve of Example-1, further comprising a filter attached to the valve housing at the vent opening.

Example-15 The suction valve of Example-1, further comprising: a valve spool disposed within the valve housing, the valve spool defining a valve spool passageway that connects the probe opening in fluid communication with the suction source opening, the valve spool being movable selectively to a home position and a depressed position relative to the valve housing; a plug on the valve spool, the plug providing a greater obstruction to the vent opening in the depressed position than in the home position; and a spring disposed within the valve housing, the spring urging the valve spool to the home position.

Example-16 A suction valve for controlling a fluid flowing through an endoscope that includes a flexible tubular probe extending from a handle body, the endoscope being connectable to a suction source, the suction valve being configurable selectively to a suction configuration and a vented configuration, the suction valve comprising: a valve housing to be supported by the handle body; a probe opening to be connected in fluid communication with the flexible tubular probe, the probe opening being of a first size and at least partially defined by the valve housing; a vent opening in fluid communication with a surrounding atmosphere when the suction valve is in the vented configuration, the vent opening being of a second size and at least partially defined by the valve housing, the vent opening being selectively open and closed, the suction valve being in the vented configuration when the vent opening is open, the suction valve being in the suction configuration when the vent opening is closed, and a suction source opening to be connected in fluid communication with the suction source, the suction source opening being of a third size and at least partially defined by the valve housing, the second size being larger than the first size and smaller than the third size, most of the fluid flowing through the suction valve flows from the vent opening to the suction source opening when the vent opening is open in the vented configuration, most of the fluid flowing through the suction valve flows from the probe opening to the suction source opening when the vent opening is closed in the suction configuration.

Example-17 The suction valve of Example-16, wherein the suction valve remains totally stationary relative to the handle body as the suction valve changes between the suction configuration and the vented configuration.

Example-18 The suction valve of Example-16, wherein the probe opening is connected in fluid communication with both the suction source opening and the vent opening in both the vented configuration and the suction configuration.

Example-19 The suction valve of Example-16, further comprising a closure attached to the valve housing at the vent opening, wherein the closure is movable between a more-closed position and an less-closed position, the closure providing a greater obstruction to the vent opening in the more-closed position than in the less-closed position.

Example-20 A suction valve system for conveying a fluid and being configurable to a suction configuration and a vented configuration, the suction valve system comprising: a handle body of an endoscope; a valve housing supported by the handle body, the valve housing at least partially defining a probe opening, a vent opening, and a suction source opening; the probe opening being in fluid communication with the suction source opening and the vent opening in both the suction configuration and the vented configuration, the vent opening being selectively open and closed, the suction valve system being in the suction configuration when the vent opening is closed, the suction valve system being in the vented configuration with the vent opening being in fluid communication with a surrounding atmosphere when the vent opening is open, the probe opening having a first flow coefficient, the vent opening having a second flow coefficient when the vent opening is open, and the suction source opening having a third flow coefficient; the second flow coefficient being greater than the first flow coefficient and less than the third flow coefficient, most of the fluid flowing through the suction valve system flows from the vent opening to the suction source opening when the vent opening is open in the vented configuration, most of the fluid flowing through the suction valve flows from the probe opening to the suction source opening when the vent opening is closed in the suction configuration; an umbilicus attached to the handle body to connect a suction source to the suction source opening of the valve housing; a flexible tubular probe connected to the handle body; and a suction tube attached to the valve housing at the probe opening and extending through the flexible tubular probe.

The disclosure should not be considered limited to the particular examples described above. Various modifications, equivalent processes, as well as numerous structures to which the disclosure can be applicable will be readily apparent to those of ordinary skill in the art upon review of the instant specification.

1. A suction valve for controlling a fluid flowing through an endoscope that includes a flexible tubular probe extending from a handle body, the endoscope being connectable to a suction source, the suction valve being configurable selectively to a suction configuration and a vented configuration, the suction valve comprising:
a valve housing to be supported by the handle body;
a probe opening with a first flow coefficient to be connected in fluid communication with the flexible tubular probe, the probe opening being at least partially defined by the valve housing;
a vent opening with a second flow coefficient in fluid communication with a surrounding atmosphere when the suction valve is in the vented configuration, the vent opening being at least partially defined by the valve housing, the vent opening being selectively open and closed, the suction valve being in the vented configuration when the vent opening is open, the suction valve being in the suction configuration when the vent opening is closed, and
a suction source opening with a third flow coefficient to be connected in fluid communication with the suction source, the suction source opening being defined by the valve housing, the second flow coefficient being greater than the first flow coefficient and less than the third flow coefficient, most of the fluid flowing through the suction valve flows from the vent opening to the suction source opening when the vent opening is open in the vented configuration, most of the fluid flowing through the suction valve flows from the probe opening to the suction source opening when the vent opening is closed in the suction configuration.

2. The suction valve of claim 16 wherein the probe opening has a first minimum diameter, the vent opening has a second minimum diameter, the suction source opening has a third minimum diameter, and the second minimum diameter is larger than the first minimum diameter and smaller than the third minimum diameter.

3. The suction valve of claim 1, wherein the second flow coefficient is closer in value to the third flow coefficient than to the first flow coefficient.

4. The suction valve of claim 1, wherein the suction valve remains totally stationary relative to the handle body as the suction valve changes between the suction configuration and the vented configuration.

5. The suction valve of claim 1, wherein the probe opening remains open in both the vented configuration and the suction configuration.

6. The suction valve of claim 1, wherein the probe opening is connected in fluid communication with both the suction source opening and the vent opening in both the vented configuration and the suction configuration.

7. The suction valve of claim 1, wherein the valve housing comprises an outer shell containing a valve cartridge, and the valve cartridge connects the probe opening in fluid communication with both the suction source opening and the vent opening in both the vented configuration and the suction configuration.

8. The suction valve of claim 7, wherein the valve cartridge remains stationary relative to the outer shell as the suction valve changes between the suction configuration and the vented configuration.

9. The suction valve of claim 7, wherein the valve cartridge is axisymmetrical.

10. The suction valve of claim 1, further comprising a closure attached to the valve housing at the vent opening.

11. The suction valve of claim 10, wherein the closure is movable between a more-closed position and an less-closed position, the closure providing a greater obstruction to the vent opening in the more-closed position than in the less-closed position.

12. The suction valve of claim 11, wherein at least part of the closure is rotatable between the more-closed position and the less-closed position.

13. The suction valve of claim 12, wherein the closure is an iris valve.

14. The suction valve of claim 1, further comprising a filter attached to the valve housing at the vent opening.

15. The suction valve of claim 1, further comprising:
a valve spool disposed within the valve housing, the valve spool defining a valve spool passageway that connects the probe opening in fluid communication with the suction source opening, the valve spool being movable selectively to a home position and a depressed position relative to the valve housing;
a plug on the valve spool, the plug providing a greater obstruction to the vent opening in the depressed position than in the home position; and
a spring disposed within the valve housing, the spring urging the valve spool to the home position.

16. A suction valve for controlling a fluid flowing through an endoscope that includes a flexible tubular probe extending from a handle body, the endoscope being connectable to a suction source, the suction valve being configurable selectively to a suction configuration and a vented configuration, the suction valve comprising:
a valve housing to be supported by the handle body;
a probe opening to be connected in fluid communication with the flexible tubular probe, the probe opening being of a first size and at least partially defined by the valve housing;
a vent opening in fluid communication with a surrounding atmosphere when the suction valve is in the vented configuration, the vent opening being of a second size and at least partially defined by the valve housing, the vent opening being selectively open and closed, the suction valve being in the vented configuration when the vent opening is open, the suction valve being in the suction configuration when the vent opening is closed, and
a suction source opening to be connected in fluid communication with the suction source, the suction source opening being of a third size and at least partially defined by the valve housing, the second size being larger than the first size and smaller than the third size, most of the fluid flowing through the suction valve flows from the vent opening to the suction source opening when the vent opening is open in the vented configuration, most of the fluid flowing through the suction valve flows from the probe opening to the suction source opening when the vent opening is closed in the suction configuration.

17. The suction valve of claim 16, wherein the suction valve remains totally stationary relative to the handle body as the suction valve changes between the suction configuration and the vented configuration.

18. The suction valve of claim 16, wherein the probe opening is connected in fluid communication with both the suction source opening and the vent opening in both the vented configuration and the suction configuration.

19. The suction valve of claim 16, further comprising a closure attached to the valve housing at the vent opening, wherein the closure is movable between a more-closed position and an less-closed position, the closure providing a greater obstruction to the vent opening in the more-closed position than in the less-closed position.

20. A suction valve system for conveying a fluid and being configurable to a suction configuration and a vented configuration, the suction valve system comprising:
a handle body of an endoscope;
a valve housing supported by the handle body, the valve housing at least partially defining a probe opening, a vent opening, and a suction source opening; the probe opening being in fluid communication with the suction source opening and the vent opening in both the suction configuration and the vented configuration, the vent opening being selectively open and closed, the suction valve system being in the suction configuration when the vent opening is closed, the suction valve system being in the vented configuration with the vent opening being in fluid communication with a surrounding atmosphere when the vent opening is open, the probe opening having a first flow coefficient, the vent opening having a second flow coefficient when the vent opening is open, and the suction source opening having a third flow coefficient; the second flow coefficient being greater than the first flow coefficient and less than the third flow coefficient, most of the fluid flowing through the suction valve system flows from the vent opening to the suction source opening when the vent opening is open in the vented configuration, most of the fluid flowing through the suction valve flows from the probe opening to the suction source opening when the vent opening is closed in the suction configuration;
an umbilicus attached to the handle body to connect a suction source to the suction source opening of the valve housing;
a flexible tubular probe connected to the handle body; and
a suction tube attached to the valve housing at the probe opening and extending through the flexible tubular probe.
